(19) United States
(12) Patent Application Publication
(10) Pub. No.: US 2026/0224376 A1
(43) Pub. Date: Aug. 6, 2026

(54) SELF-ADJUSTING SOCKET FOR LOWER LIMB PROSTHESIS

(71) Applicant: Vessl Prosthetics Inc., Ilderton (CA)

(72) Inventors: Oleksiy ZAIKA, Ilderton (CA); Sydney Motz ROBINSON, London (CA)

(21) Appl. No.: 19/148,981

(22) PCT Filed: Jan. 16, 2024
(86) PCT No.: PCT/CA2024/050040

§ 371 Date: Jul. 17, 2025

Related U.S. Application Data
(60) Provisional application No. 63/440,212, filed on Jan. 20, 2023.

(51) Int. Cl. A61F 2/80

(52) U.S. Cl. CPC A61F 2/80

(57) ABSTRACT

A socket for a lower limb prosthesis comprises a receptacle for a residuum, an anterior fluidic actuator disposed on an anterior side of the receptacle and a posterior fluid-driven compensator disposed on a posterior side of the receptacle. The anterior fluidic actuator is in fluid communication with the posterior fluid-driven compensator through a fluidic linkage and, upon application of anterior outward pressure thereto, drives fluid through the fluidic linkage toward the posterior fluid-driven compensator, which has a posterior fitting that is movable inwardly and outwardly relative to the receptacle and moves inwardly upon supply of the fluid to the posterior fluid-driven compensator. A control valve in the fluidic linkage permits fluid from the anterior fluidic actuator to reach the posterior fluid-driven compensator and inhibits fluid from flowing back toward the anterior fluidic actuator.

### CROSS-REFERENCE TO RELATED APPLICATION

This application claims priority to U.S. Provisional Patent Application No. 63/440,212 filed on Jan. 20, 2023.

### TECHNICAL FIELD

The present disclosure relates to lower limb prostheses, and more particularly to sockets for lower limb prostheses.

### BACKGROUND

Amputation of a limb is tragic. Fortunately, medical technology has advanced considerably, and a wide range of prosthetic limbs are now available.

A prosthetic limb typically consists of a prosthetic socket, an alignment device, one or more pylons, and an end effector. The prosthetic socket interfaces with the residual limb, or residuum, and connects it to the rest of the prosthetic limb. The alignment device typically maintains proper alignment between the socket and the rest of the prosthesis. The pylon(s) connect the socket and/or alignment device to the end effector. There may be a single pylon (e.g. for transtibial and transradial amputees) or multiple pylons (e.g. for transfemoral and transhumeral amputees) pylon(s) that connect the socket and/or alignment device to the end effector. The end effector typically replicates a foot or hand, depending on whether the prosthesis is for an upper limb (transhumeral, transradial) or lower limb (transfemoral, transtibial) amputee. In the case of a transfemoral amputee, the prosthetic limb also typically includes a knee joint.

Arguably the most important component of a prosthetic limb is the prosthetic socket. It is the sole component connecting the residuum to the rest of the prosthetic limb. Effective interface (fit) between the socket and the residuum is crucial.

A major complication in achieving an effective interface between the socket and the residuum is the fact that the size and shape of the residuum is not constant, but fluctuates over time. These fluctuations include short term fluctuations and long term fluctuations.

Following amputation, the edema, or swelling, decreases and muscles in the residuum may atrophy from disuse, which leads to significant changes in the residuum's volume.

These are examples of relatively long term fluctuations, which may be accommodated straightforwardly, for example by the expedient of taking periodic measurements. In the acute phase following amputation (approximately two years post-amputation), an amputee typically requires several “check sockets” which are simple sockets that are used to check whether the fit is appropriate. Since the residuum loses significant volume from edema and muscle atrophy, amputees require a series of check sockets until their residuum volume has become sufficiently stable and does not decrease as significantly week-to-week.

Unfortunately, change in the volume of the residuum is not only an acute issue following amputation, but often persists throughout an amputee's life. Moreover, short term changes in volume are common, and the volume of the residuum can change considerably over the course of a single day or even a few hours. Factors that can affect the volume of the residuum include, but are not limited to, exercise, diet, lifestyle, and other comorbidities, as well as weather.

Since conventional prosthetic sockets are rigid and unchanging in size and shape, a change in the residuum's volume alters the socket fit, that is, the interface between the socket and residuum. Typically, an amputee will progressively lose volume over the long term, as a result of edema reduction and muscle loss, and the volume will oscillate over the short term. Activities of daily living, which include any kind of ambulation, can drive fluid out of the limb, reducing its volume.

Prosthetic socks may be used to accommodate the longer term decreases in volume-more socks and/or thicker socks may be used as residuum volume decreases over time. However, prosthetic socks are not well suited to accommodate the shorter term fluctuations in residuum volume, as they would require the amputee to remove their prosthetic limb, add socks on top of their residuum, and then reattach the prosthetic limb. Adding or removing prosthetic socks is extremely disruptive to an amputee's activities of daily living; they must sit down to remove their prosthetic limb and rearrange or remove articles of clothing to access their residuum and add or remove prosthetic socks appropriately. They must also bring socks with them to every destination in case the need to add or remove prosthetic socks arises. Typically, amputees must add several prosthetic socks (in some cases, over 10) to properly account for the volume they lost in their residuum.

Furthermore, even if adding or removing socks throughout the day were practical, prosthetic socks can only compensate for a finite amount of volume change, and do not accommodate changes in the shape of the residuum that may result from the volume changes. As a result, painful forces can act on a part or parts of the residuum (particularly those areas with bony protrusions).

It has been observed that daily fluctuations in residuum volume for a femoral residuum or a tibial residuum typically occur at the posterior of the residuum. One attempt to address the daily fluctuations in residuum volume is described in U.S. Pat. No. 7,655,049 to Phillips, which describes a prosthetic device having a socket with an insert having a bladder system for monitoring and compensating for volume fluctuations in a residual limb. A plurality of bladders are preferably provided, in one embodiment, substantially only on a posterior portion of the socket. The bladders may be organized into zones, with the zones being inflatable to differing pressures depending on volume fluctuations in a residual limb. Pressure sensors may be provided for each bladder or for each zone, and flow regulators may be provided to control fluid flow into or out of the bladders or zones of bladders based on readings from the pressure sensors to control volume within the insert. Alternatively, bladders can be manually inflated depending on an amputee's needs.

As can be imagined, this system requires either complex sensors and electronic arrangements, which result in increased complexity and cost, or manual adjustment, which increases the inconvenience for the amputee.

### SUMMARY

In one aspect, a socket for a lower limb prosthesis comprises a receptacle for a residuum, an anterior fluidic actuator disposed on an anterior side of the receptacle, and a posterior fluid-driven compensator disposed on a posterior side of the receptacle. The anterior fluidic actuator is in fluid communication with the posterior fluid-driven compensator through a fluidic linkage extending between the anterior fluidic actuator and the posterior fluid-driven compensator. The anterior fluidic actuator is adapted, upon application of anterior outward pressure thereto, to drive a fluid through the fluidic linkage toward the posterior fluid-driven compensator. The posterior fluid-driven compensator includes a posterior fitting that is movable inwardly and outwardly relative to the receptacle and adapted to move inwardly relative to the receptacle upon supply to the posterior fluid-driven compensator of the fluid driven by the anterior fluidic actuator through the fluidic linkage. At least one control valve is disposed within the fluidic linkage, interposed between the anterior fluidic actuator and the posterior fluid-driven compensator. The control valve(s) having at least a first configuration in which the control valve(s) permit the fluid driven by the anterior fluidic actuator to reach the posterior fluid-driven compensator and to inhibit the fluid from flowing from the posterior fluid-driven compensator toward the anterior fluidic actuator. With the control valve(s) in the first configuration, application of pressure to the anterior fluidic actuator drives the fluid through the hydraulic linkage into posterior fluidic compensator.

In an embodiment, the anterior fluidic actuator includes an anterior fitting that is movable inwardly and outwardly relative to the receptacle and adapted to move outwardly relative to the receptacle upon application of the anterior outward pressure thereto, and the anterior fluidic actuator, the fluidic linkage and the posterior fluid-driven compensator are configured such that upon supply to the posterior fluid-driven compensator of the fluid driven by the anterior fluidic actuator through the fluidic linkage, inward anterior movement of the posterior fitting exceeds anterior displacement of the anterior fitting.

In an embodiment, the control valve(s) comprise at least one one-way valve.

In an embodiment, the control valve(s) are configurable between at least two configurations including the first configuration and a second configuration. In a particular embodiment, in the second configuration, the control valve(s) permit the fluid to flow from the posterior fluid-driven compensator toward the anterior fluidic actuator when a posterior outward pressure applied to the posterior fluid-driven compensator exceeds the anterior outward pressure applied to the anterior fluidic actuator. In some embodiments, the control valve(s) may be movable between the first configuration and the second configuration by an external switch. In some embodiments, the control valve(s) may have a third configuration which valve vents the fluid to ambient. In such embodiments, the control valve(s) may be movable between the first configuration, the second configuration and the third configuration by an external switch.

In an embodiment, the anterior fluidic actuator is a hydraulic actuator, the posterior fluid-driven compensator is a hydraulic compensator, and the fluidic linkage is a hydraulic linkage. In such an embodiment, the hydraulic linkage is preferably a direct hydraulic linkage omitting any intermediate reservoir for the fluid.

In an embodiment, the anterior fluidic actuator is a pneumatic actuator, the posterior fluid-driven compensator is a pneumatic compensator, and the fluidic linkage is a pneumatic linkage.

In an embodiment, the anterior fluidic actuator comprises at least one anterior piston-cylinder assembly and the posterior fluid-driven compensator comprises at least one posterior piston-cylinder assembly.

In an embodiment, the anterior fluidic actuator comprises at least one anterior bladder, and the posterior fluid-driven compensator comprises at least one posterior bladder.

In an embodiment, one of the anterior fluidic actuator and the posterior fluid-driven compensator comprises at least one piston-cylinder assembly and the other of the anterior fluidic actuator and the posterior fluid-driven compensator comprises at least one bladder.

A prosthetic limb may comprise a socket as described above. The prosthetic limb may be, for example, a transfemoral prosthesis or a transtibial prosthesis.

In an embodiment, the posterior fluid-driven compensator comprises a plurality of individual compression elements.

In an embodiment, the anterior fluidic actuator, the posterior fluid-driven compensator and the fluidic linkage form a pumpless fluid transfer circuit.

In another aspect, a socket for a lower limb prosthesis comprises a receptacle for a residuum, an anterior mechanical actuator disposed on an anterior side of the receptacle, a posterior mechanical compensator disposed on an anterior side of the receptacle, and a mechanical linkage extending between the anterior mechanical actuator and the posterior mechanical compensator. The anterior mechanical actuator includes an anterior fitting that is movable inwardly and outwardly relative to the receptacle and adapted to move outwardly relative to the receptacle upon application of anterior outward pressure thereto. The posterior mechanical compensator includes a posterior fitting being movable inwardly and outwardly relative to the receptacle. The anterior mechanical actuator, the posterior mechanical compensator and the mechanical linkage cooperate to transfer anterior outward pressure applied to the anterior mechanical actuator through the mechanical linkage to the posterior mechanical compensator to cause the posterior mechanical compensator to move inwardly relative to the receptacle. The mechanical linkage includes a gearing mechanism whereby an anterior component of inward movement of the posterior mechanical compensator exceeds an anterior component of outward movement of the anterior mechanical actuator.

### BRIEF DESCRIPTION OF THE DRAWINGS

These and other features will become more apparent from the following description in which reference is made to the appended drawings wherein:

FIG. 1 is a schematic representation of a first illustrative embodiment of a socket for a lower limb prosthesis;

FIG. 2 is a top perspective view of an illustrative, non-limiting physical embodiment of a socket according to the design of the socket shown schematically in FIG. 1;

FIG. 3 is a bottom perspective view of the socket of FIG. 2;

FIG. 4 is a first side elevation view of the socket of FIG. 2;

FIG. 5 is a second side elevation view of the socket of FIG. 2;

FIG. 6 is a third side elevation view of the socket of FIG. 2;

FIG. 7 is a fourth side elevation view of the socket of FIG. 2;

FIG. 8 is a top plan view of the socket of FIG. 2;

FIG. 9 is a bottom plan view of the socket of FIG. 2;

FIG. 10 is a partially exploded bottom perspective view of the socket of FIG. 2 with fluidic linkage tubing thereof removed;

FIG. 11 is a partially exploded bottom perspective view of the socket of FIG. 2 with the fluidic linkage tubing thereof intact;

FIG. 12 is a side cross-sectional view of the socket of FIG. 2;

FIG. 13 is a side partial interior view of the socket of FIG. 2;

FIGS. 14A and 14B are top plan views of the socket of FIG. 2 showing interaction thereof with a residuum;

FIG. 15 is a top plan view of a second illustrative embodiment of a socket for a lower limb prosthesis, showing interaction thereof with a residuum; and

FIG. 16 is a schematic representation of a third illustrative embodiment of a socket for a lower limb prosthesis.

### DETAILED DESCRIPTION

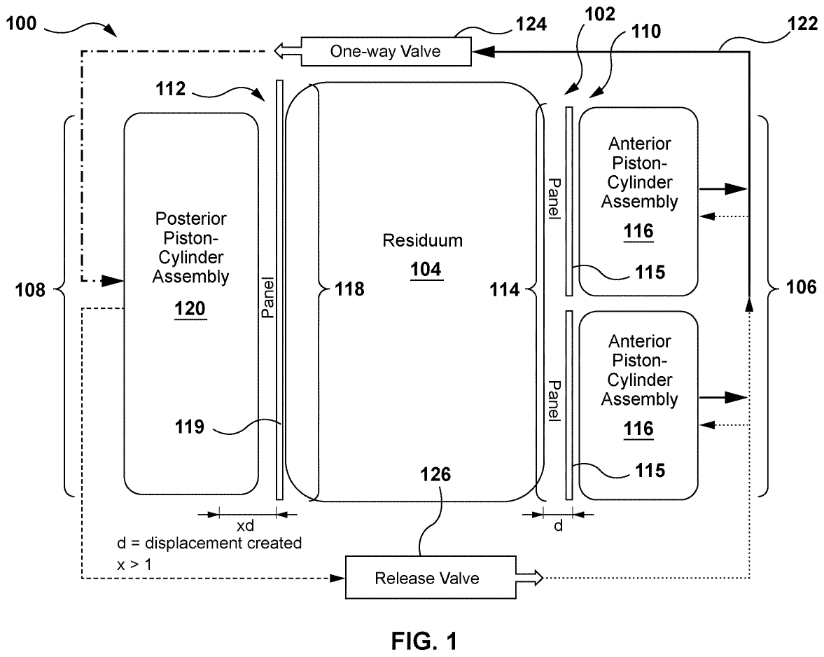

Reference is now made to FIG. 1, which is a schematic representation of a socket, indicated generally by reference 100, for a lower limb prosthesis. The lower limb prosthesis may be, for example, a transfemoral prosthesis or a transtibial prosthesis. Thus, sockets as described herein may be incorporated into a complete prosthetic limb.

As noted above, daily fluctuations in residuum volume for a femoral residuum or a tibial residuum typically occur at the posterior of the residuum, with shrinkage of the residuum creating a gap between the residuum and the posterior interior surface of the socket and resulting in misfit and potentially pistoning of the limb within the socket space. The socket 100 is configured to automatically adjust to such shrinkage and obviate the misfit.

It has been observed that with shrinkage of the residuum, along with the gap between the residuum and the posterior interior surface of the socket there is also resultant pressure on an anterior interior surface of the socket. With any slight bend in the knee, the forces accumulate at the anterior and when the amputee is walking, there is a forward lean that creates pressure on the anterior section. If there is wiggle room inside the socket, aside from pistoning, there is an anterior tilt of the limb inside the socket during gait. The socket 100 is configured to use this anterior pressure to trigger adjustments that reduce or eliminate the posterior gap.

Continuing to refer to FIG. 1, the socket 100 comprises a receptacle 102 for a residuum 104, as well as an anterior fluidic actuator 106 and a posterior fluid-driven compensator 108. The term “fluid”, as used herein, refers to a liquid or a gas. Preferably an incompressible liquid is used, although in some embodiments a compressible fluid, such as a gas, may be used. Correspondingly, the term “fluidic actuator” refers to a device that drives a fluid-flow solely by mechanical (including pneumatic and/or hydraulic) action, and excludes electronic or electromechanical sensors that cause an electrical signal to drive a pump, solenoid or other electronic component. Similarly, the term “fluid-driven compensator” refers to a device which is driven solely by fluidic (pneumatic and/or hydraulic) action, and excludes devices driven by electronic or electromechanical action, such as an electric pump or solenoid that drives a fluid.

The anterior fluidic actuator 106 is disposed on an anterior side 110 of the receptacle 102, and the posterior fluid-driven compensator 108 is disposed on a posterior side 112 of the receptacle 102. The terms “anterior” and “posterior”, as used in this context, refer to the anatomical directions of the residuum 104 that is accommodated by the receptacle 102.

In the illustrated embodiment, the anterior fluidic actuator 106 comprises an anterior fitting 114 disposed on the anterior side 110 of the receptacle 102 and mechanically coupled to two anterior piston-cylinder assemblies 116, preferably single-acting piston cylinder assemblies. FIG. 1 shows the anterior fitting 114 as comprising two spaced-apart anterior panels 115, with a gap between the panels 115 to accommodate the tibia (shin bone); some embodiments may have only a single panel, or three or more panels, which may be separate or may be flexibly coupled to one another. Moreover, while FIG. 1 shows flat panels 115 for purposes of schematic illustration, the panels may be curved and/or may be custom contoured to conform to the shape of the residuum 104.

Similarly, in the illustrated embodiment the posterior fluid-driven compensator 108 comprises a posterior fitting 118 mechanically coupled to a posterior piston-cylinder assembly 120, preferably a single-acting piston-cylinder assembly. FIG. 1 shows the posterior fitting 118 as comprising a posterior panel. While illustrated for simplicity as a single flat posterior panel 119, the posterior fitting 118 may comprise two or more panels, which may be separate or may be flexibly coupled to one another, and may be curved and/or custom contoured to conform to the shape of the residuum 104. Thus, in some embodiments the posterior fluid-driven compensator comprises a plurality of individual compression elements.

The anterior fitting 114 is movable inwardly and outwardly relative to the receptacle 102, with corresponding movement of the anterior pistons within the anterior cylinders of the anterior piston-cylinder assemblies 116 according to the mechanical linkage. For example, the panels 115 of the anterior fitting 114 may be movable inwardly and outwardly relative to the receptacle 102. Similarly, the posterior fitting 118 is movable inwardly and outwardly relative to the receptacle 102, with corresponding movement of the posterior piston within the posterior cylinder of the posterior piston-cylinder assembly 120. For example, the panel 119 of the posterior fitting 118 may be movable inwardly and outwardly relative to the receptacle 102.

The anterior fluidic actuator 106 is in fluid communication with the posterior fluid-driven compensator 108 through a fluidic linkage 122 extending between the anterior fluidic actuator 106 and the posterior fluid-driven compensator 108. More particularly, the anterior piston-cylinder assemblies 116 are in fluid communication with the posterior piston-cylinder assembly 120 through the fluidic linkage 122.

A compensation valve 124 is disposed within the fluidic linkage 122, interposed between the anterior fluidic actuator 106 and the posterior fluid-driven compensator 108, more particularly between the anterior piston-cylinder assemblies 116 and the posterior piston-cylinder assembly 120. While a single compensation valve 124 is shown for simplicity of illustration, in some embodiments there may be a plurality of compensation valves (e.g. a multi-line fluidic linkage with one compensation valve for each line).

In the illustrated embodiment, the compensation valve 124 is a one-way valve. Specifically, the compensation valve 124 is oriented to permit the fluid driven by the anterior fluidic actuator 106, specifically the anterior piston-cylinder assemblies 116, to reach the posterior fluid-driven compensator 108, specifically the posterior piston-cylinder assembly 120, and to inhibit the fluid from flowing from the posterior fluid-driven compensator 108 toward the anterior fluidic actuator 106. Other valve configurations are also contemplated, as described further below.

The anterior fluidic actuator 106 is adapted, upon application of anterior outward pressure thereto with suitable valve configuration (described further below), to drive the fluid through the fluidic linkage 122 toward the posterior fluid-driven compensator 108. More particularly, anterior outward pressure applied to the anterior fitting 114 causes outward movement of the anterior fitting 114, which acts through the mechanical linkage to initiate at least a partial return stroke of the anterior pistons within the anterior cylinders of the anterior piston-cylinder assemblies 116. This return stroke drives fluid from the anterior cylinders of the anterior piston-cylinder assemblies 116 into the fluidic linkage 122, past the compensation valve 124 and into the posterior cylinders of the posterior piston-cylinder assembly 120, initiating at least a partial extension stroke of the posterior piston within the posterior cylinder of the posterior piston-cylinder assembly 120. This extension stroke drives anterior inward movement of the posterior fitting 118 toward the residuum 104 by way of the mechanical linkage between the posterior piston-cylinder assembly 120 and the posterior fitting 118. Thus, the posterior fitting 118 is adapted to move inwardly relative to the receptacle 102 upon supply to the posterior fluid-driven compensator 108 of the fluid driven by the anterior fluidic actuator 106 through the fluidic linkage 122.

Of note, the anterior fluidic actuator 106, the fluidic linkage 122 and the posterior fluid-driven compensator 108 are configured such that upon supply to the posterior fluid-driven compensator 108 of the fluid driven by the anterior fluidic actuator 106 through the fluidic linkage 122, the anterior component of the inward movement of the posterior fitting 118 exceeds the anterior component of the outward movement of the anterior fitting 114. This is illustrated in FIG. 1 by the arrow “d” showing the outward displacement of the anterior fitting 114, and the arrow “xd” showing the inward displacement of the posterior fitting 118, where x>1. In the illustrated embodiment, this differential displacement is achieved by the use of two anterior piston-cylinder assemblies 116 and only a single posterior piston-cylinder assembly 120 so that, for identical piston-cylinder assemblies, the single posterior piston-cylinder assembly 120 will receive approximately twice as much fluid as is discharged by each of the anterior piston-cylinder assemblies 116. This is merely one technique for achieving differential displacement, and other techniques may also be used, for example by varying the volume and or/number of individual piston-cylinder assemblies to achieve a desired differential displacement. Thus, in other embodiments there may be different numbers of posterior piston-cylinder assemblies and/or anterior piston-cylinder assemblies. Suitable configuration of the piston-cylinder assemblies is within the capability of one of ordinary skill in the art, now informed by the present disclosure.

As noted above, in the illustrated embodiment, the compensation valve 124 is a one-way valve that permits the fluid driven by the anterior fluidic actuator 106 to reach the posterior fluid-driven compensator 108 and inhibits the fluid from flowing from the posterior fluid-driven compensator 108 toward the anterior fluidic actuator 106. The socket 100 further comprises a release valve 126 interposed between the anterior fluidic actuator 106 and the posterior fluid-driven compensator 108, more particularly between the anterior piston-cylinder assemblies 116 and the posterior piston-cylinder assembly 120. The release valve 126 is ordinarily closed, that is, biased into a closed position, and is provided with an external switch, for example a lever, button or other switch element, for selectively moving the release valve 126 into an open position. Optionally, the release valve 126 may be a one-way valve oriented opposite to the compensation valve 124 such that in the open position, the release valve 126 permits permit the fluid driven by the posterior fluid-driven compensator 108 to reach the anterior fluidic actuator 106 and to inhibit the fluid from flowing from the anterior fluidic actuator 106 toward the posterior fluid-driven compensator 108. Optionally, the release valve 126 may be a pressure relief valve, so as to prevent pressure in the posterior piston-cylinder assembly 120 from exceeding a preset maximum, with excess pressure being bled back to the anterior piston-cylinder assemblies 116. In one non-limiting embodiment, the preset maximum may be 8 kPa. In the closed position, the release valve 126 inhibits fluid flow in both directions. The compensation valve 124 and the release valve 126 are control valves, and together are configurable (by opening or closing the release valve 126) between a first configuration and a second configuration. The first configuration (release valve 126 closed) permits the fluid driven by the anterior fluidic actuator 106 to reach the posterior fluid-driven compensator 108 and inhibits the fluid from flowing from the posterior fluid-driven compensator 108 toward the anterior fluidic actuator 106. The second configuration (release valve 126 open) permits the fluid driven by the posterior fluid-driven compensator 108 to reach the anterior fluidic actuator 106.

Accordingly, when the control valves (compensation valve 124 and release valve 126) are in the first configuration, application of pressure to the anterior fluidic actuator 106 drives the fluid through the hydraulic linkage 122 into the posterior hydraulic compressor 108. Conversely, when the control valves 124, 126 are in the second configuration, the control valves 124, 126 permit the fluid to flow from the posterior fluid-driven compensator 108 toward the anterior fluidic actuator 106 when the posterior outward pressure applied to the posterior fluid-driven compensator 108 exceeds the anterior outward pressure applied to the anterior fluidic actuator 106. Thus, by using the external switch to open the release valve 126, and applying pressure to the posterior fluid-driven compensator 108, fluid can be returned from the posterior fluid-driven compensator 108 to the anterior fluidic actuator 106.

While multiple valves are shown for simplicity of illustration, in other embodiments a single valve may be provided which is movable between the first configuration and the second configuration, for example by one or more external switches. Optionally, the second configuration may also inhibit the fluid from flowing from the anterior fluidic actuator toward the posterior fluid-driven compensator. Additionally, the socket 100 may optionally be provided with valving having a third configuration in which the valving vents the fluid to ambient. This may be provided by a third (or second) valve, or a single valve may be movable between the first, second and third configuration. In any of these embodiments, the valve(s) may be configured to be movable between the first configuration, the second configuration and the third configuration by respective individual external switches, or by a single multi-position switch.

As can be seen in FIG. 1, the anterior fluidic actuator 106, the posterior fluid-driven compensator 108 and the fluidic linkage 122 form a pumpless fluid transfer circuit; fluid transfer results solely from pressure applied to the fittings 114, 118. As noted above, the term “fluidic actuator” refers to a device that drives a fluid-flow solely by mechanical (including pneumatic and/or hydraulic) action, and the term “fluid-driven compensator” refers to a device which is driven solely by fluidic (pneumatic and/or hydraulic) action.

In some embodiments, the anterior fluidic actuator 106, including the anterior fitting 114 and the anterior piston-cylinder assemblies 116, is a hydraulic actuator and the posterior fluid-driven compensator 108, including the posterior fitting 118 and posterior piston-cylinder assembly 120 is a hydraulic compensator. Thus, in such embodiments the anterior piston-cylinder assemblies 116 and the posterior piston-cylinder assembly 120 are hydraulic piston-cylinder assemblies, and the fluidic linkage 122 is a hydraulic linkage. Of note, as can be seen in FIG. 1, where the fluidic linkage 122 is a hydraulic linkage, the fluidic linkage 122 is a direct hydraulic linkage omitting any intermediate reservoir for the fluid.

In other embodiments, the anterior fluidic actuator 106, including the anterior fitting 114 and the anterior piston-cylinder assemblies 116, is a pneumatic actuator and the posterior fluid-driven compensator 108, including the posterior fitting 118 and posterior piston-cylinder assembly 120 is a pneumatic compensator. Thus, in such embodiments the anterior piston-cylinder assemblies 116 and the posterior piston-cylinder assembly 120 are pneumatic piston-cylinder assemblies, and the fluidic linkage 122 is a pneumatic linkage.

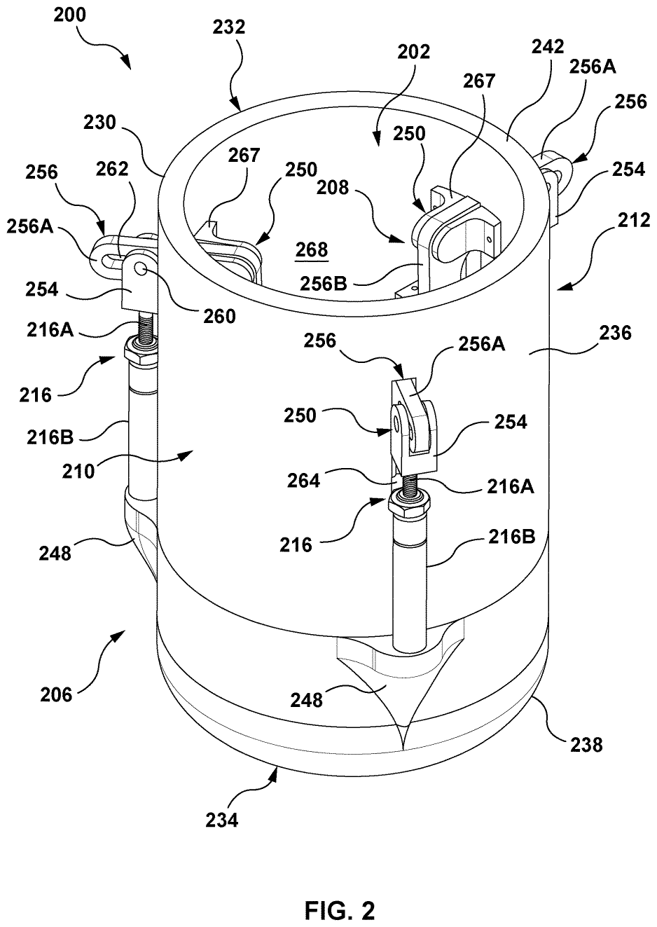

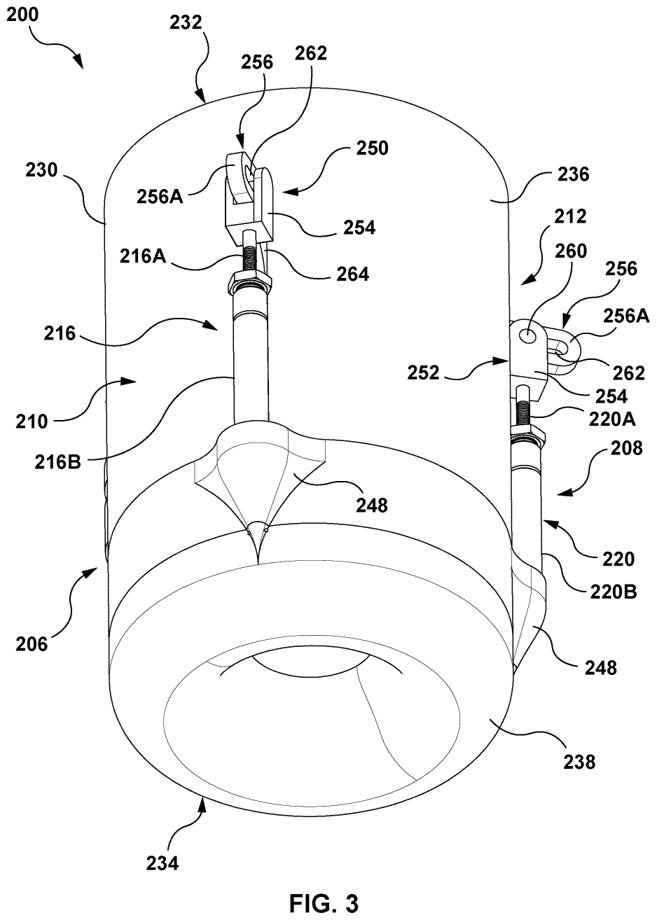

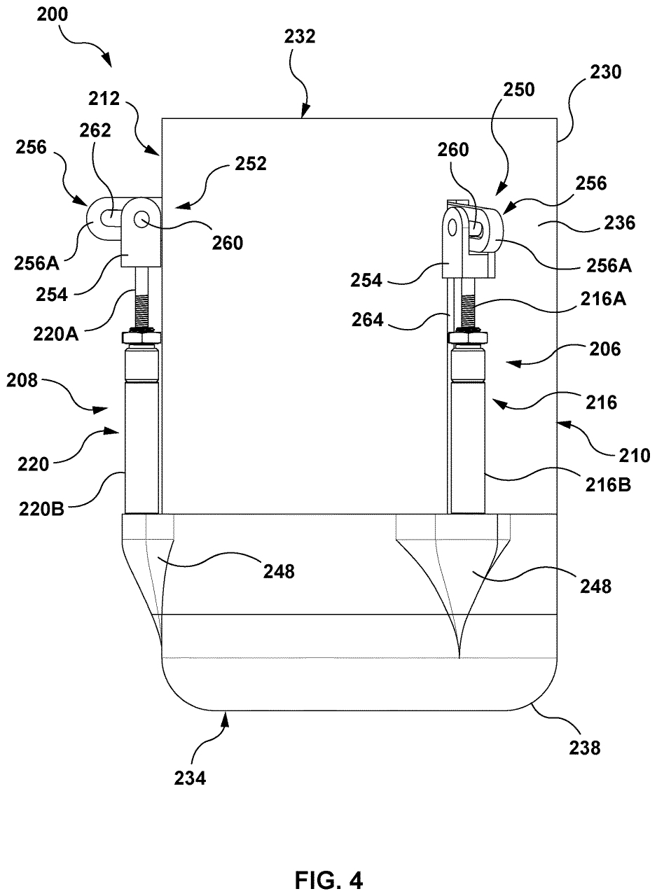

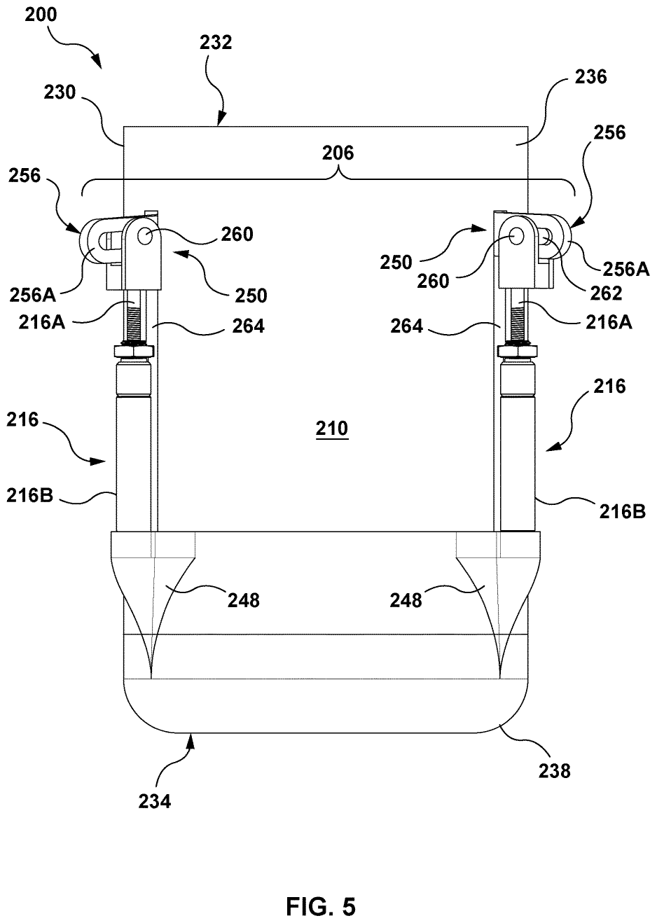

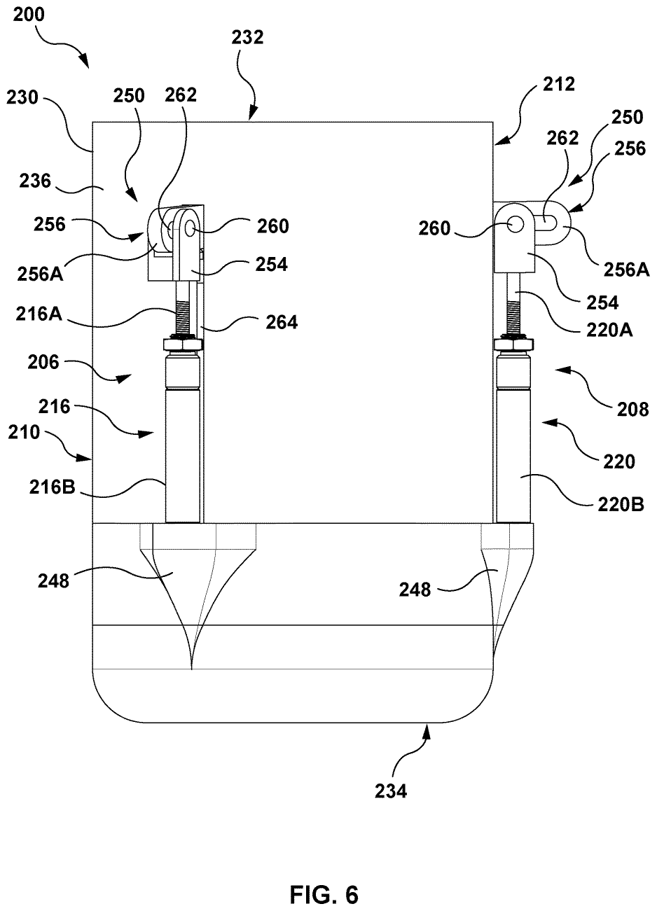

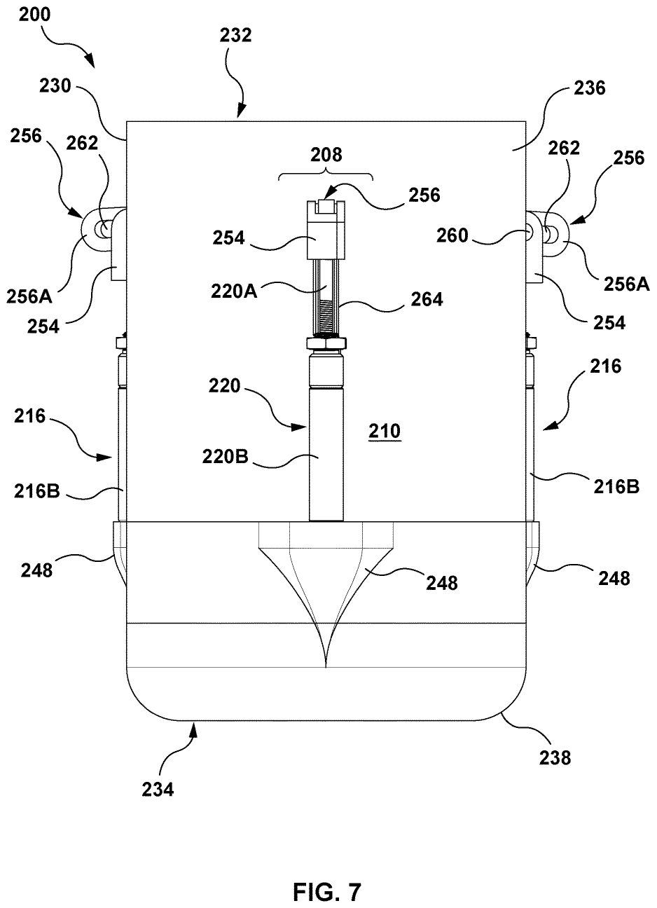

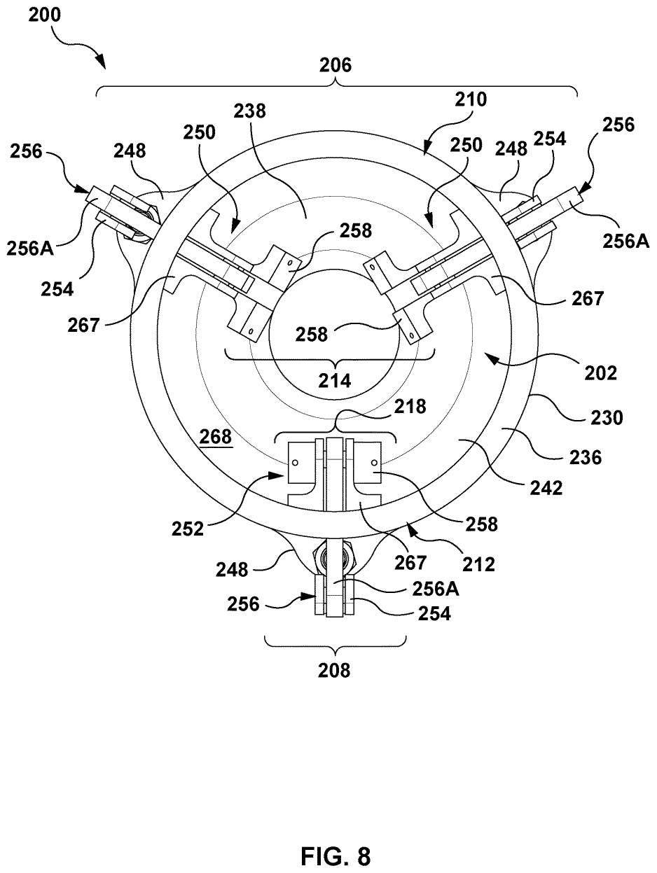

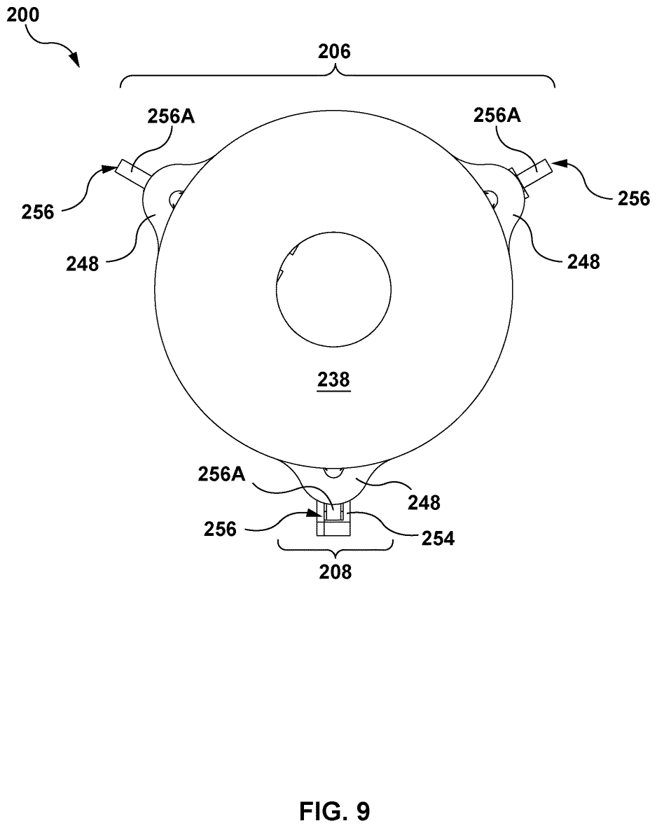

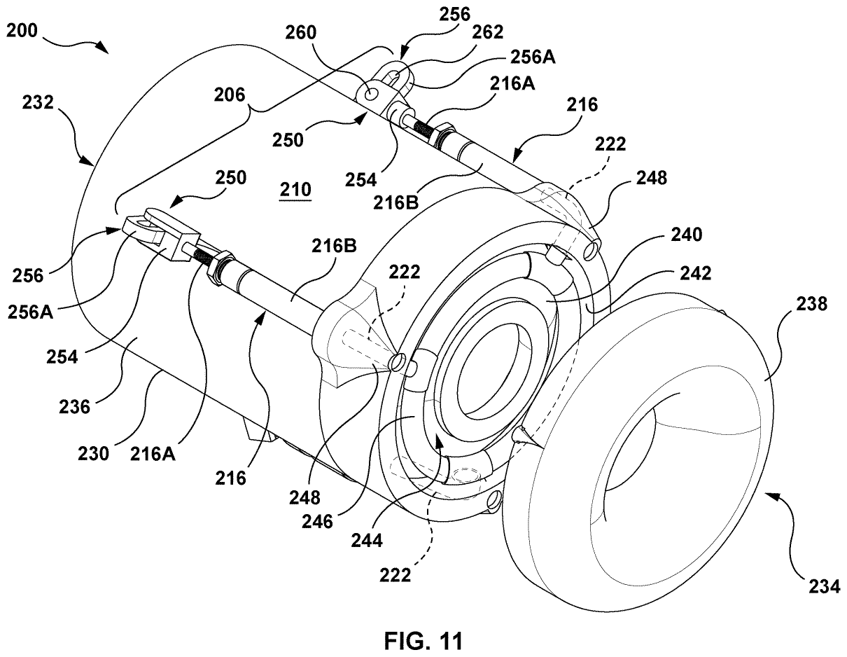

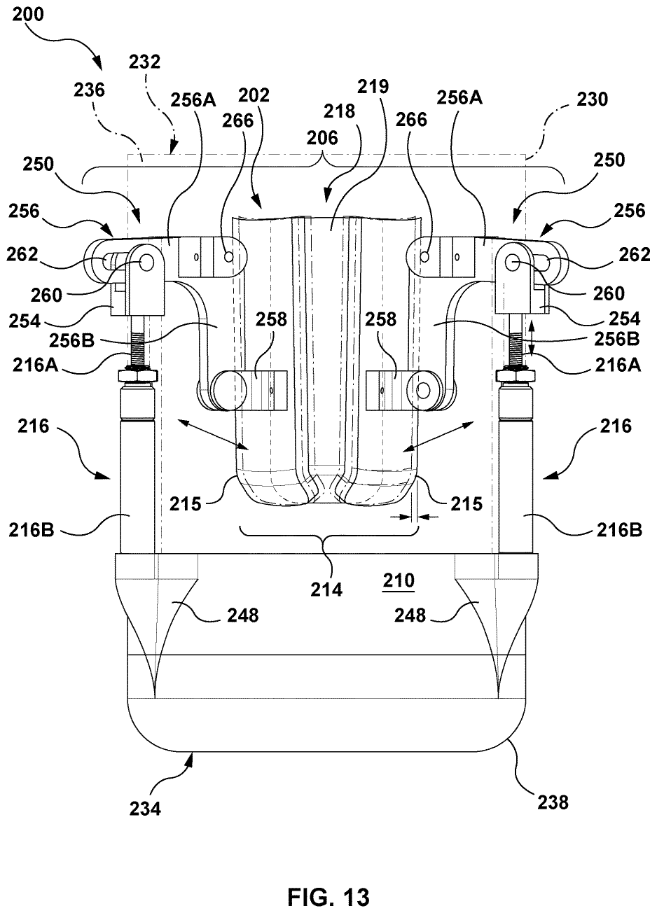

FIGS. 2 to 14B show an illustrative, non-limiting physical embodiment 200 of a socket according to the design of the socket 100 shown schematically in FIG. 1. Accordingly, in FIGS. 2 to 14B like reference numerals refer to corresponding components, but with the prefix “2” rather than “1” at the hundreds place value in the reference numeral.

The socket 200 shown in FIGS. 2 to 14B comprises a hollow generally cylindrical housing 230 having a residuum end 232 and a prosthetic end 234 opposite the residuum end 232. The housing 230 is open at the residuum end 232 to form the receptacle 202. The prosthetic end 234 is adapted to securely receive a prosthetic limb. Any suitable mounting arrangement may be used. The housing 230 may be made from any suitable material, for example carbon fibre, a suitable polymer or a suitable metal or metal alloy, with lighter weight being preferred.

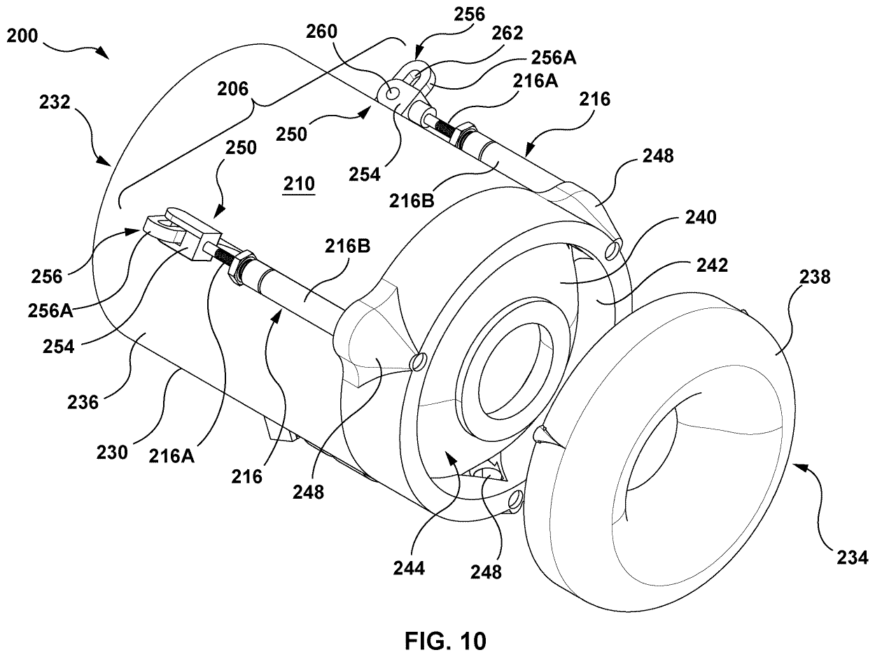

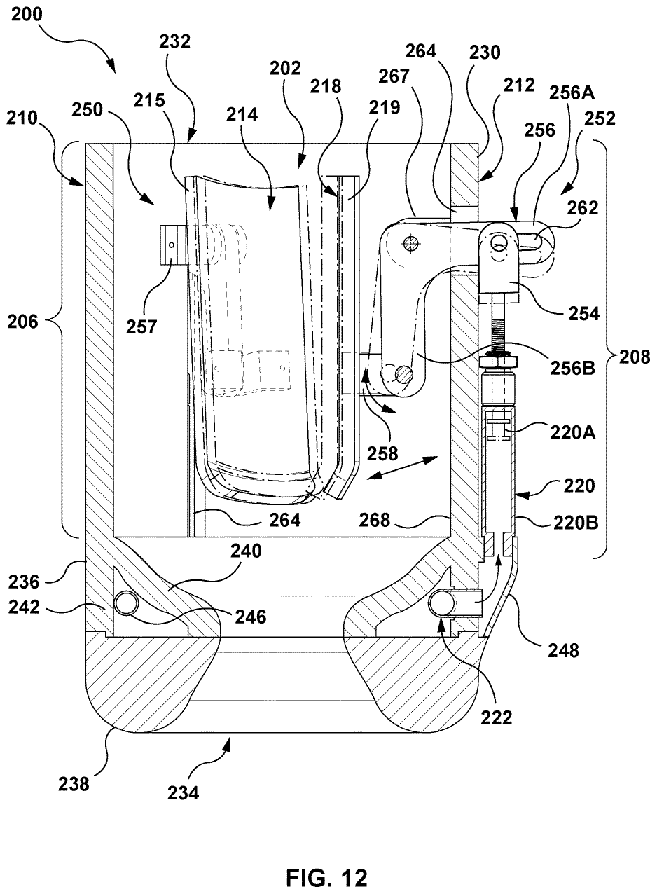

As best seen in FIGS. 10 to 12, the housing 230 comprises a substantially cylindrical main body 236 and a removable annular end cap 238. The main body 236 and the end cap 238 may be formed from the same material or from different materials. As best shown in FIG. 12, the main body 236 has an inwardly projecting ogee-shaped annular shoulder 240 adjacent the prosthetic end 234 of the housing 230; the annular shoulder 240 cooperates with the body wall 242 of the main body 236 to form an annular channel 244 at the end of the main body 236 adjacent the prosthetic end 234 of the housing 230. Tubing 246 forming part of the fluidic linkage 222 is disposed within the annular channel 244 and enclosed by the end cap 238. Hollow fluid couplings 248 extend outwardly from the main body 236 adjacent the prosthetic end 234 of the housing 230. The fluid couplings 248 enable fluid communication between the tubing 246 and the piston-cylinder assemblies 216, 220 to complete the fluidic linkage 222. Removal of the end cap 238 allows access to the tubing 246 and valves (e.g. control valves 124. 126) for servicing, such as adding or removing fluid to control pressurization of the system. The piston-cylinder assemblies 216, 220 are disposed on the exterior of the body wall 242 of the main body 236, and arranged to extend substantially parallel to a longitudinal axis of the main body 236. This is merely one illustrative embodiment; in other embodiments, for example, the body wall may be hollow and the piston cylinder assemblies and/or fluid couplings may disposed wholly or partially within the body wall. Other suitable arrangements are also contemplated.

In the illustrated embodiment, the fluid couplings 248 and piston-cylinder assemblies 216, 220 are arranged at intervals, with the anterior piston-cylinder assemblies 216 arranged on the anterior side 210 of the receptacle 202 and the posterior piston-cylinder assembly arranged on the posterior side 212 of the receptacle 202. In the illustrated embodiment, the fluid couplings 248 and piston-cylinder assemblies 216, 220 are equally spaced at 120 degree intervals as shown, but this is merely one illustrative embodiment, and not limiting. Other embodiments may have different spacing.

The anterior fluidic actuator 206 comprises an anterior fitting 214 formed by two curved anterior panels 215 (see FIGS. 12 to 14B) and further comprises the two anterior piston-cylinder assemblies 216, which are arranged in circumferential registration with respective ones of the anterior panels 215. The anterior panels 215 are disposed within the housing 230, inside the receptacle 202 on the anterior side 210 thereof. The anterior piston-cylinder assemblies 216 are disposed outside of the housing 230, and therefore outside of the receptacle 202. Thus, the anterior fluidic actuator 206 is disposed on the anterior side 210 of the receptacle 202, partially within the receptacle 202 and partially outside the receptacle 202. In other embodiments the anterior fluidic actuator may be disposed entirely within the receptacle.

Similarly, the posterior fluid-driven compensator 208 comprises a posterior fitting 218 formed by a single curved posterior panel 219 (see FIGS. 12 to 14B) and the single posterior piston-cylinder assembly 220, which is in circumferential registration with the posterior panel 219. The posterior panel 219 is disposed within the housing 230, inside the receptacle 202 on the posterior side 212 thereof. The posterior piston-cylinder assembly 220 is disposed outside of the housing 230, and therefore outside of the receptacle 202. Thus, the posterior fluid-driven compensator 208 is disposed on the posterior side 212 of the receptacle 202, partially within the receptacle 202 and partially outside the receptacle 202. In other embodiments the posterior fluidic compensator may be disposed entirely within the receptacle.

The anterior panels 215 and the posterior panel 219 may be made from any suitable material, and are preferably custom-fitted to the contours of the residuum of the user. In some embodiments, the anterior panels 215 and the posterior panel 219 may each comprise a rigid panel base of one or more standard sizes, that is, with no particular contouring for an individual user, with a prosthetic liner custom fitted to the user disposed on the panel base.

In the illustrated embodiment respective mechanical couplings are interposed between the panels 215, 219 and the respective pistons 216A, 220A of the respective piston-cylinder assemblies 216, 220. Thus, there is a respective anterior mechanical coupling 250 between each anterior panel 215 and the respective anterior piston 216A, and a posterior mechanical coupling 252 between the posterior panel 219 and the posterior piston 220A. In FIGS. 2 to 11, the anterior panels 215 and posterior panel 219 are removed to provide a better view of the components of the mechanical couplings 250, 252; the anterior panels 215 and posterior panel 219 are shown in FIGS. 12 through 14B.

As best seen in FIGS. 12 to 14B, in the illustrated embodiment, each mechanical coupling 250, 252 comprises a piston bracket 254, a generally L-shaped bell crank 256 and a panel bracket 258 configured so that longitudinal movement of the respective pistons 216A, 220A within the respective cylinders 216B, 220B corresponds to inward and outward movement of the respective panels 215, 219 relative to the receptacle 102. In the illustrated embodiment, this inward and outward movement will be substantially radial movement relative to the substantially cylindrical main body 236; there is also an axial component of the movement relative to the main body 236 although this is substantially smaller than the radial component. The mechanical couplings 250, 252 are merely illustrative and not limiting, and other types of mechanical coupling may also be used; one alternate example is briefly described below.

More particularly, in the illustrated embodiment the piston bracket 254 includes a guide pin 260 that rides within a guide slot 262 disposed toward the end of the piston arm 256A of the bell crank 256, and the panel bracket 258 is pivotally coupled to the distal end of the panel arm 256B of the bell crank 256; that is, the end remote from the piston arm 256A. The bell crank 256 itself is positioned with its panel arm 256B and part of the piston arm 256A disposed within the receptacle 102 and the remainder of the piston arm 256A extending through a longitudinally extending slot 264 formed in the main body 236. In the illustrated embodiment this slot 264 extends from adjacent the residuum end 232 of the housing 230 to the annular shoulder 240 and can be used to facilitate easier installation of the bell crank 256, with the majority of the slot 264 being covered by one of the piston-cylinder assemblies 216, 220 after installation. The bell crank 256 is pivotally coupled to the main body 236 by way of a crank bracket 267 so as to be pivotable within the slot 264 by way of a pivot pin 266 extending through the crank bracket 267 and through the bell crank 256 between the piston arm 256A and the panel arm 256B thereof.

Thus, as best seen in FIG. 12, in operation, when the control valves (e.g. control valves 124, 126 in FIG. 1) are in the first configuration, outward pressure applied to the anterior panels 215 acts, through the respective panel brackets 258, bell cranks 256 and piston brackets 254 to drive retraction of the respective anterior pistons 216A into the respective anterior cylinders 216B, forcing fluid through the tubing 246, past the compensation valve, and into the posterior cylinder 220B. This in turn forces the posterior piston 220A into extension, which acts, through the respective piston bracket 254, bell crank 256 and panel bracket 258, to drive inward movement of the posterior panel 219. By suitable configuration of the anterior piston-cylinder assemblies 216 and posterior piston-cylinder assembly 220, the anterior component of the inward movement of the posterior panel 219 will exceed the anterior displacement, that is, the anterior component of the outward movement, of the anterior panels 215. This will tighten the receptacle 202 to compensate for the residuum shrinkage that produced the pressure on the anterior fitting 214.

Conversely, when the control valves (e.g. control valves 124, 126 in FIG. 1) are in the second configuration, outward pressure applied to the posterior panel 219 that exceeds the anterior outward pressure applied to the anterior panels 215 acts, through the respective panel brackets 258, bell cranks 256 and piston brackets 254 to drive retraction of the posterior piston 220A into the posterior cylinder 220B. This forces fluid through the tubing 246, past the release valve, and into the posterior cylinder 220B. This, in turn, forces the anterior pistons 216A into extension, which acts, through the respective piston bracket 254, bell crank 256 and panel bracket 258, to drive inward movement of the anterior panels 215. Because of the configuration of the anterior piston-cylinder assemblies 216 and posterior piston-cylinder assembly 220, the anterior component of the outward movement of the posterior panel 219 will exceed the anterior displacement, that is, the anterior component of the inward movement, of the anterior panels 215. This will loosen the receptacle 202, for example to relieve excessive pressure on the residuum, or to remove the prosthesis.

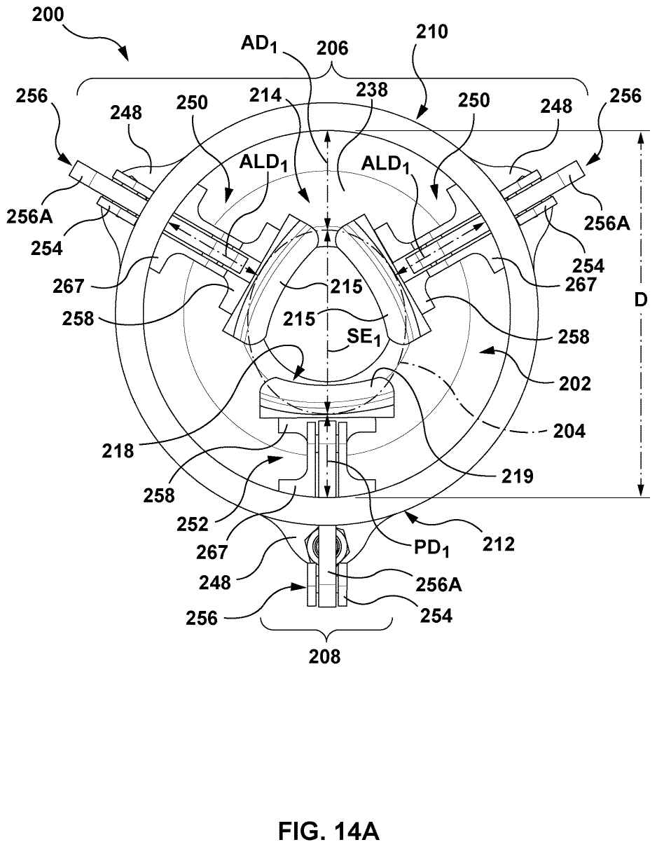

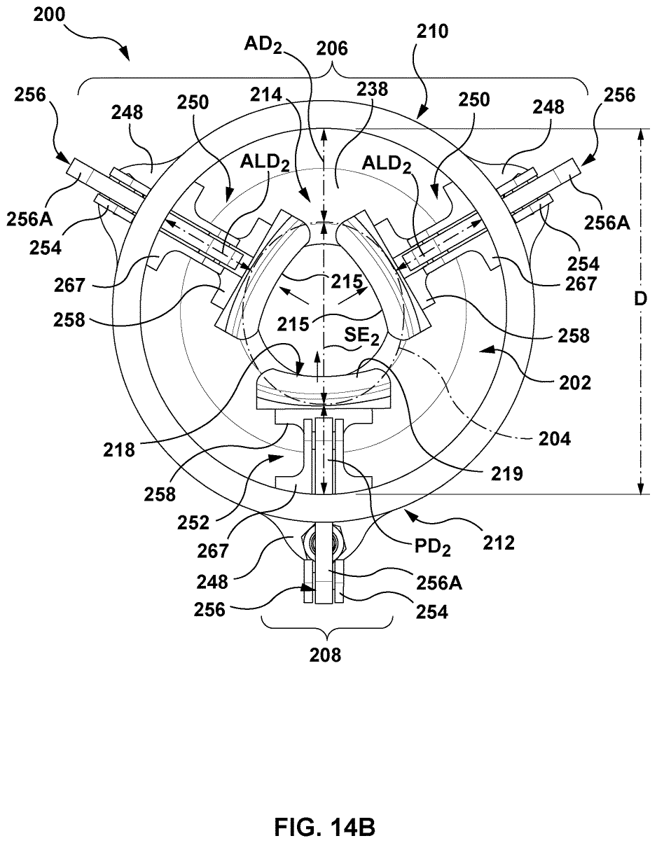

Reference is now made to FIGS. 14A and 14B. As shown in FIG. 14A, in an initial state after installing the prosthesis, such as at the beginning of the day before any substantial shrinkage of the residuum 204 has occurred, the residuum 102 has an initial circumference and an initial sagittal extent SE₁ (the thickness of the residuum as measured in the sagittal plane), with the residuum 204 trapped between the anterior panels 215 and the posterior panel 219. The anterior piston-cylinder assemblies 216, posterior piston-cylinder assembly 220 and fluidic linkage 222 are initially pressurized to a desired pressure. As can be seen in FIG. 14A, at the initial circumference and initial sagittal extent SE₁ the residuum 204 has an initial anterior distance AD₁ from the interior surface 268 of the main body 236 of the housing 230, and has an initial posterior distance PD₁ from the interior surface 268 of the main body 236. The sum of the initial sagittal extent RE₁, the initial anterior distance AD₁ and the initial posterior distance PD₁ is equal to the internal diameter D of the main body 236. In the illustrated embodiment, the anterior panels 215 and their respective panel brackets 258, bell cranks 256 and piston brackets 254 are arranged substantially in the anterolateral directions, relative to the residuum 204. The initial anterolateral distance between the residuum 204 and the interior surface 268 of the main body 236, that is, distance measured in the anterolateral directions, is denoted by ALD₁.

As noted above, with shrinkage of the residuum 204, there is pressure on an anterior interior surface of the socket resulting from the amputee tending to lean forward to improve his or her balance to compensate for the posterior gap. This leaning applies pressure to the anterior panels 215, causing outward movement thereof, which, as described above, and assuming that the pressure through the compensation valve does not exceed the preset maximum, leads to inward movement of the posterior panel 219, resulting in the configuration shown in FIG. 14B.

After shrinkage of the residuum 204, the residuum 204 has a reduced circumference and a reduced sagittal extent SE₂, which is less than the initial sagittal extent SE₁ and the posterior distance between the residuum 204 and the interior surface 268 has increased from the initial posterior distance PD₁ to a shrinkage posterior distance PD₂ where PD₁<PD₂. The leaning pressure applied to the anterior panels 215 has displaced the anterior panels 215 anterolaterally. This reduces the anterolateral distance between the residuum 204 and the interior surface 268 from the initial anterolateral distance ALD₁ to a compensating anterolateral distance ALD₂ where ALD₁>ALD₂. Thus, ALD₁>ALD₂ where the change in anterolateral distance exceeds the change in anterior distance. Outward displacement of the anterior panels 215 is in the anterolateral directions, and includes an anterior component, which reduces the anterior distance between the residuum 204 and the interior surface 268 from the initial anterior distance AD₁ to a compensating anterior distance AD₂ where AD₁>AD₂. The outward (anterolateral) movement of the anterior panels 215 drives fluid from the anterior piston-cylinder assemblies 216 through the fluidic linkage 222 into the posterior piston-cylinder assembly 220, which drives inward movement of the posterior panel 219. This inward movement compensates for the increase in posterior distance from PD₁ to PD₂. As noted above, the inward movement of the posterior panel 219 exceeds the anterior displacement of the anterior panel 215 (the anterior component of the outward anterolateral movement of the anterior panels 215). Thus, while AD₁>AD₂, it is also the case that PD₂>PD₁ by an amount greater than AD₁>AD₂, i.e. (AD₁−AD₂)<(PD₂−PD₁), so that:

$$D \approx {AD}_{1} + {SE}_{1} + {PD}_{1} \approx {AD}_{2} + {SE}_{2} + {PD}_{2}$$

whereby the inward movement of the posterior panel 219 (PD₂−PD₁) compensates for the shrinkage of the residuum 204 (SE₁−SE₂) as well as the anterior component of the outward movement of the anterior panels 215 (AD₁−AD₂).

In the embodiment shown in FIGS. 2 to 14B, the piston-cylinder assemblies 216, 220 are arranged to extend substantially parallel to a longitudinal axis of the main body 236, with mechanical couplings 250, 252 comprising a bell crank 256 used to redirect longitudinal movement of the respective pistons 216A, 220A within the respective cylinders 216B, 220B to cause inward and outward movement of the respective panels 215, 219 relative to the receptacle 102. As noted above, mechanical couplings 250, 252 are merely illustrative and not limiting. For example, in other embodiments, the piston-cylinder assemblies may be arranged to extend substantially perpendicularly to the longitudinal axis of the main body (i.e. radially outwardly) so that movement of the pistons corresponds directly to inward and outward movement of the respective panels, without the need for the bell crank.

Additionally, embodiments are contemplated in which the anterior piston-cylinder assemblies 216 and/or the posterior piston-cylinder assembly 220 and associated mechanical couplings 250, 252 are replaced by one or more bladders in fluid communication with the fluidic linkage. Thus, in one embodiment, the anterior fluidic actuator comprises at least one anterior bladder and the posterior fluid-driven compensator comprises at least one posterior bladder assembly.

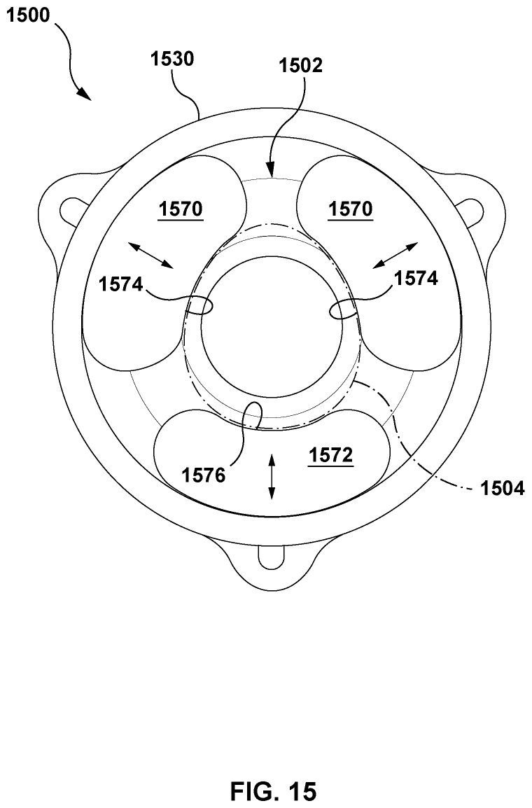

FIG. 15 shows an illustrative socket 1500 comprising a hollow generally cylindrical housing 1530 similar to the socket 200 shown in FIGS. 2 to 14B and having a receptacle 1502. Instead of the anterior piston-cylinder assemblies 216, posterior piston-cylinder assembly 220 and associated mechanical couplings 250, 252 in the socket 200 as shown in FIGS. 2 to 14B, the socket 1500 shown in FIG. 15 comprises two anterior bladders 1570 and a posterior bladder 1572. The anterior bladders 1570 may be connected in fluid communication with the posterior bladder 1572 by a fluidic linkage (not shown) similar to the fluidic linkage 122, 222 described above. In the illustrated embodiment, the bladders 1570, 1572 are generally reniform when viewed from above so as to embrace the residuum 1504; this is merely illustrative and not limiting.

In an embodiment in which bladders are used, the fittings may comprise the inwardly facing surfaces of the bladders. Thus, in the illustrative socket 1500 shown in FIG. 15, in which the anterior fluidic actuator comprises two anterior bladders 1570, the inwardly facing surfaces 1574 of the anterior bladders 1570 form an anterior fitting that is movable inwardly and outwardly relative to the receptacle 1502 and adapted to move outwardly relative to the receptacle upon application of the anterior outward pressure thereto. Similarly, in the illustrative socket 1500 shown in FIG. 15, the posterior fluid-driven compensator comprises a posterior bladder 1572 and the inwardly facing surface 1576 of the posterior bladder 1572 form a posterior fitting that is movable inwardly and outwardly relative to the receptacle 1502 and adapted to move inwardly relative to the receptacle 1502 upon supply to the posterior bladder 1572 of the fluid driven by the anterior bladders 1570 through the fluidic linkage. The surfaces 1574, 1576 of the bladders 1570, 1572, engage the residuum 1504. The bladders 1570, 1572 are configured such that upon supply to the posterior bladder 1572 of the fluid driven by the anterior bladders 1570 through the fluidic linkage, inward movement (anterior displacement) of the inwardly facing surface 1576 of the posterior bladder 1572 exceeds the anterior displacement of the inwardly facing surfaces 1574 of the anterior bladders 1570.

The size, number and position of the bladders 1570, 1572 shown in FIG. 15 is merely illustrative, and different sizes, numbers and positions of bladders may be used.

It is also contemplated that a combination of one or more bladders and one or more piston-cylinder assemblies may be used. For example, in some embodiments one of the anterior fluidic actuator and the posterior fluid-driven compensator comprises at least one piston-cylinder assembly and the other of the anterior fluidic actuator and the posterior fluid-driven compensator comprises at least one bladder.

The foregoing description relates to embodiments of a socket for a lower limb prosthesis including a receptacle for a residuum, with an anterior fluidic actuator disposed on an anterior side of the receptacle and a posterior fluid-driven compensator disposed on a posterior side of the receptacle. In addition to the foregoing fluidic embodiments, aspects of the present disclosure are also directed to mechanical embodiments of a self-adjusting socket.

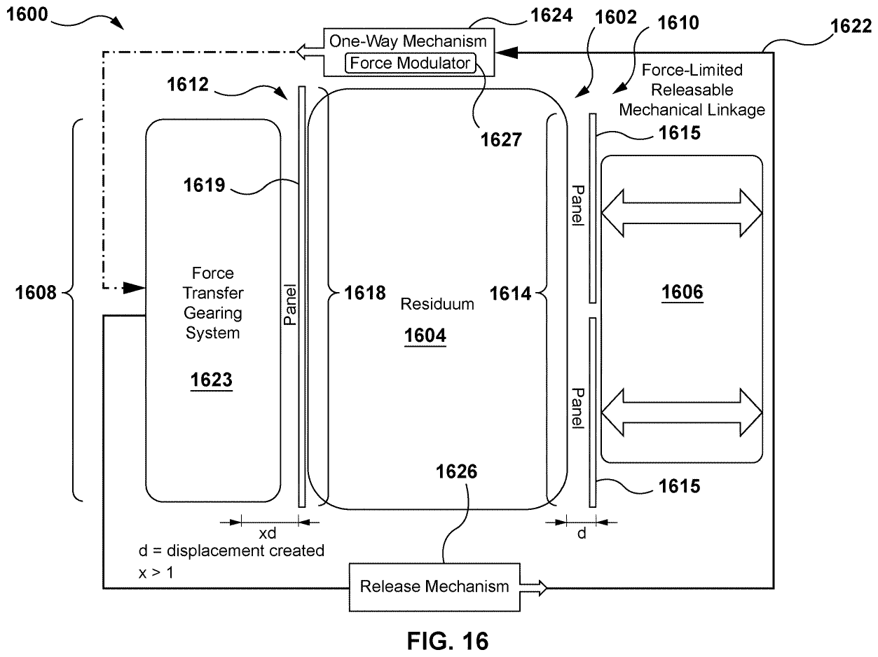

Reference is now made to FIG. 16, which shows another embodiment of a socket 1600 for a lower limb prosthesis. The socket 1600 comprises a receptacle 1602 for a residuum 1604, as well as an anterior mechanical actuator 1606 and a posterior mechanical compensator 1608.

The anterior mechanical actuator 1606 is disposed on an anterior side 1610 of the receptacle 1602, and the posterior mechanical compensator 1608 is disposed on a posterior side 1612 of the receptacle 1602. The terms “anterior” and “posterior”, as used in this context, refer to the anatomical directions of the residuum 1604 that is accommodated by the receptacle 1602.

In the illustrated embodiment, the anterior mechanical actuator 1606 comprises an anterior fitting 1614 disposed on the anterior side 1610 of the receptacle 1602. FIG. 16 shows the anterior fitting 1614 as comprising two spaced-apart anterior panels 1615, with a gap between the panels 1615 to accommodate the tibia (shin bone); some embodiments may have only a single panel, or three or more panels, which may be separate or may be flexibly coupled to one another. Moreover, while FIG. 16 shows flat panels 1615 for purposes of schematic illustration, the panels may be curved and/or may be custom contoured to conform to the shape of the residuum 1604. Similarly, the posterior mechanical compensator 1608 comprises a posterior fitting 1618 shown for illustrative purposes as comprising a posterior panel 1619. While illustrated for simplicity as a single flat posterior panel 1619, the posterior fitting 1618 may comprise two or more panels, which may be separate or may be flexibly coupled to one another, and may be curved and/or custom contoured to conform to the shape of the residuum 1604.

The anterior fitting 1614 is movable inwardly and outwardly relative to the receptacle 1602, e.g. panel(s) 1615 of the anterior fitting 1614 may be movable inwardly and outwardly relative to the receptacle 1602. Similarly, the posterior fitting 1618 is movable inwardly and outwardly relative to the receptacle 1602, e.g. panel(s) 1619 of the posterior fitting 1618 may be movable inwardly and outwardly relative to the receptacle 1602.

A force-limited releasable mechanical linkage 1622 extends between the anterior mechanical actuator 1606 and the posterior mechanical compensator 1608. In one non-limiting example, the mechanical linkage 1622 may comprise a timing belt or similar structure extending circumferentially around the receptacle 1602. The mechanical linkage 1622 includes a force transfer gearing system 1623, and the anterior mechanical actuator 1606, the posterior mechanical compensator 1608 and the mechanical linkage 1622 cooperate to transfer anterior outward pressure applied to the anterior mechanical actuator 1606 (e.g. pressure on the anterior fitting 1614) through the mechanical linkage 1622 to the posterior mechanical compensator 1608 to cause the posterior mechanical compensator 1608 (e.g. the posterior fitting 1618) to move inwardly relative to the receptacle 1602. The force transfer gearing system 1623 is configured so that the anterior component of inward movement of the posterior mechanical compensator 1608 (e.g. movement of the posterior fitting 1618) exceeds the anterior component of outward movement of the anterior mechanical actuator 1606 (e.g. movement of the anterior fitting 1614). This is illustrated in FIG. 16 by the arrow “d” showing the outward displacement of the anterior fitting 1614, and the arrow “xd” showing the inward displacement of the posterior fitting 1618, where x>1. The force transfer gearing system 1623 may comprise, for example, bevel gears, worm gears and/or rack and pinion gears to translate the circumferential movement of the timing belt (or similar structure) into transverse motion of the posterior panel 1619. These are merely non-limiting examples and any suitable gearing arrangement may be used; configuration of a suitable gearing arrangement will be within the capability of one of ordinary skill in the art, now informed by the present disclosure.

The mechanical linkage 1622 includes a one-way mechanism 1624, as well as a release mechanism 1626. For example, the one-way mechanism 1624 may comprise a ratchet, and the release mechanism 1626 may comprise a clutch. These are merely illustrative, non-limiting examples. The one-way mechanism 1624 and/or the release mechanism 1626 may be incorporated into the force transfer gearing system 1623, or may be distinct therefrom.

When the release mechanism 1626 is disengaged, the one-way mechanism 1624 allows mechanical force applied to the anterior mechanical actuator 1606 (e.g. pressure on the anterior fitting 1614) to be transferred through the mechanical linkage 1622 to the posterior mechanical compensator 1608 to cause the posterior mechanical compensator 1608 (e.g. the posterior fitting 1618) to move inwardly relative to the receptacle 1602. At the same time, with the release mechanism 1626 disengaged, the one-way mechanism 1624 inhibits mechanical force applied to the posterior mechanical compensator 1608 (e.g. pressure on the posterior fitting 1618) from being transferred to the anterior mechanical actuator 1606 (e.g. the anterior fitting 1614). When the release mechanism 1626 is engaged, however, the one-way mechanism 1624 allows mechanical force applied to the posterior mechanical compensator 1608 (e.g. pressure on the posterior fitting 1618) to be transferred through the mechanical linkage 1622 to the anterior mechanical actuator 1606 (e.g. the anterior fitting 1614). Preferably, the release mechanism 1626 is biased into the disengaged configuration, and may be provided with an external switch, for example a lever, button or other switch element, for selectively moving the release mechanism 1626 is biased into the engaged configuration.

Preferably, the one-way mechanism 1624 includes a force modulator 1627, which is separate and distinct from the release mechanism 1626. In one embodiment, the force modulator 1627 is configured to inhibit force beyond a specified threshold from being transferred from the anterior mechanical actuator 1606 (e.g. pressure on the anterior fitting 1614) through the mechanical linkage 1622 to the posterior mechanical compensator 1608. In another embodiment, the force modulator 1627 may be configured to allow mechanical force applied to the posterior mechanical compensator 1608 (e.g. pressure on the posterior fitting 1618) to be transferred through the mechanical linkage 1622 to the anterior mechanical actuator 1606 (e.g. the anterior fitting 1614) if a pressure threshold is exceeded. The use of a force modulator can inhibit excessive force from being applied to the posterior of the residuum 1604, for example to avoid having the ground reaction force incurred from sprinting or jumping transferred from the anterior mechanical actuator 1606 through the mechanical linkage 1622 to the posterior mechanical compensator 1608.

As the residuum 1604 loses volume, which is typical throughout the day and in the long term, and the amputee is weight bearing, the residuum applies pressure to the anterior fitting 1614 (e.g. anterior panels 1615). The socket 1600 adjusts to the loss in residuum volume by transferring the anterior force of the residuum on the anterior fitting 1614 (e.g. anterior panels 1615) through the mechanical linkage 1622 to the posterior fitting 1618 (e.g. posterior panels 1619) and the posterior area of the residuum 1604. Thus, the socket 1600 maintains proper socket fit with the residuum 1604.

Certain illustrative embodiments have been described by way of example. It will be apparent to persons skilled in the art that a number of variations and modifications can be made without departing from the scope of the claims.

### LISTING OF REFERENCE NUMERALS

The following list of reference numerals is provided for convenience of reference, and no limitation is implied.

### FIG. 1

100 Socket
102 Receptacle
104 Residuum
106 Anterior fluidic actuator
108 Posterior fluid-driven compensator
110 Anterior side of receptacle
112 Posterior side of receptacle
114 Anterior fitting
115 Anterior panels
116 Anterior piston-cylinder assemblies
118 Posterior fitting
119 Posterior panel
120 Posterior piston-cylinder assembly
122 Fluidic linkage
124 Compensation valve
126 Release valve

### FIGS. 2-14B

200 Socket
202 Receptacle
206 Anterior fluidic actuator
208 Posterior fluid-driven compensator
210 Anterior side of receptacle
212 Posterior side of receptacle
214 Anterior fitting
215 Anterior panels
216 Anterior piston-cylinder assemblies
216A Anterior pistons
216B Anterior cylinders
218 Posterior fitting
219 Posterior panel
220 Posterior piston-cylinder assembly
220A Posterior piston
220B Posterior cylinder
222 Fluidic linkage
230 Housing
232 Residuum end
234 Prosthetic end
236 Main body of housing
238 End cap
240 Annular shoulder
242 Body wall of main body
244 Annular channel
246 Tubing
248 Fluid couplings
250 Anterior mechanical couplings
252 Posterior mechanical coupling
254 Piston bracket
256 Bell crank
256A Piston arm of bell crank
256B Panel arm of bell crank
258 Panel bracket
260 Guide pin
262 Guide slot
264 Slot in main body
266 Pivot pin
267 Crank bracket
268 Interior surface of main body

### FIGS. 14A and 14B Only

204 Residuum
AD₁ Initial anterior distance
AD₂ Compensating anterior distance
ALD₁ Initial anterolateral distance
ALD₂ Compensating anterolateral distance
D Internal diameter of main body of housing
PD₁ Initial posterior distance
PD₂ Shrinkage posterior distance
SE₁ Initial sagittal extent of residuum
SE Reduced sagittal extent of residuum

### FIG. 15

1500 Socket
1502 Receptacle
1504 Residuum
1570 Anterior bladders
1572 Posterior bladder
1574 Inwardly facing surfaces of anterior bladders
1576 Inwardly facing surfaces of posterior bladder

### FIG. 16

1600 Socket
1602 Receptacle
1604 Residuum
1606 Anterior mechanical actuator
1608 Posterior mechanical compensator
1610 Anterior side of receptacle
1612 Posterior side of receptacle
1614 Anterior fitting
1615 Anterior panels
1618 Posterior fitting
1619 Posterior panel
1622 Force-limited mechanical linkage
1623 Force transfer gearing system
1624 One-way mechanism
1626 Release mechanism
1627 Force modulator

What is claimed is:

1. A socket for a lower limb prosthesis, comprising:
a receptacle for a residuum;
an anterior fluidic actuator disposed on an anterior side of the receptacle; and
a posterior fluid-driven compensator disposed on a posterior side of the receptacle;
the anterior fluidic actuator being in fluid communication with the posterior fluid-driven compensator through a fluidic linkage extending between the anterior fluidic actuator and the posterior fluid-driven compensator;
the anterior fluidic actuator being adapted, upon application of anterior outward pressure thereto, to drive a fluid through the fluidic linkage toward the posterior fluid-driven compensator;
the posterior fluid-driven compensator including a posterior fitting that is movable inwardly and outwardly relative to the receptacle and adapted to move inwardly relative to the receptacle upon supply to the posterior fluid-driven compensator of the fluid driven by the anterior fluidic actuator through the fluidic linkage;
wherein at least one control valve is disposed within the fluidic linkage, interposed between the anterior fluidic actuator and the posterior fluid-driven compensator;
the at least one control valve having at least a first configuration in which the control valve is configured to permit the fluid driven by the anterior fluidic actuator to reach the posterior fluid-driven compensator and to inhibit the fluid from flowing from the posterior fluid-driven compensator toward the anterior fluidic actuator;
whereby, when the at least one control valve is in the first configuration, application of pressure to the anterior fluidic actuator drives the fluid through the hydraulic linkage into posterior fluidic compensator.

2. The socket of claim 1, wherein:
the anterior fluidic actuator includes an anterior fitting that is movable inwardly and outwardly relative to the receptacle and adapted to move outwardly relative to the receptacle upon application of the anterior outward pressure thereto;
the anterior fluidic actuator, the fluidic linkage and the posterior fluid-driven compensator being configured such that upon supply to the posterior fluid-driven compensator of the fluid driven by the anterior fluidic actuator through the fluidic linkage, inward anterior movement of the posterior fitting exceeds anterior displacement of the anterior fitting.

3. The socket of claim 1, wherein the at least one control valve comprises at least one one-way valve.

4. The socket of claim 1, wherein the at least one control valve is configurable between at least two configurations including the first configuration and a second configuration.

5. The socket of claim 4, wherein, in the second configuration, the at least one control valve is configured to permit the fluid to flow from the posterior fluid-driven compensator toward the anterior fluidic actuator when a posterior outward pressure applied to the posterior fluid-driven compensator exceeds the anterior outward pressure applied to the anterior fluidic actuator.

6. The socket of claim 5, wherein the at least one control valve is movable between the first configuration and the second configuration by an external switch.

7. The socket of claim 5, wherein the at least one control valve has a third configuration in which the at least one control valve vents the fluid to ambient.

8. The socket of claim 7, wherein the at least one control valve is movable between the first configuration, the second configuration and the third configuration by an external switch.

9. The socket of claim 1, wherein:
the anterior fluidic actuator is a hydraulic actuator; and
the posterior fluid-driven compensator is a hydraulic compensator; and
the fluidic linkage is a hydraulic linkage.

10. The socket of claim 9, wherein the hydraulic linkage is a direct hydraulic linkage omitting any intermediate reservoir for the fluid.

11. The socket of claim 1, wherein:
the anterior fluidic actuator is a pneumatic actuator; and
the posterior fluid-driven compensator is a pneumatic compensator; and
the fluidic linkage is a pneumatic linkage.

12. The socket of claim 1, wherein:
the anterior fluidic actuator comprises at least one anterior piston-cylinder assembly; and
the posterior fluid-driven compensator comprises at least one posterior piston-cylinder assembly.

13. The socket of claim 1, wherein:
the anterior fluidic actuator comprises at least one anterior bladder; and
the posterior fluid-driven compensator comprises at least one posterior bladder.

14. The socket of claim 1, wherein:
one of the anterior fluidic actuator and the posterior fluid-driven compensator comprises at least one piston-cylinder assembly; and
the other of the anterior fluidic actuator and the posterior fluid-driven compensator comprises at least one bladder.

15. A prosthetic limb comprising the socket of claim 1.

16. The prosthetic limb of claim 1, wherein the prosthetic limb is a transfemoral prosthesis.

17. The prosthetic limb of claim 1, wherein the prosthetic limb is a transtibial prosthesis.

18. The socket of claim 1, wherein the posterior fluid-driven compensator comprises a plurality of individual compression elements.

19. The socket of claim 1, wherein the anterior fluidic actuator, the posterior fluid-driven compensator and the fluidic linkage form a pumpless fluid transfer circuit.

20. A socket for a lower limb prosthesis, comprising:
a receptacle for a residuum;
an anterior mechanical actuator disposed on an anterior side of the receptacle;
a posterior mechanical compensator disposed on an anterior side of the receptacle; and
a mechanical linkage extending between the anterior mechanical actuator and the posterior mechanical compensator;
the anterior mechanical actuator including an anterior fitting that is movable inwardly and outwardly relative to the receptacle and adapted to move outwardly relative to the receptacle upon application of anterior outward pressure thereto;
the posterior mechanical compensator including a posterior fitting being movable inwardly and outwardly relative to the receptacle;
the anterior mechanical actuator, the posterior mechanical compensator and the mechanical linkage cooperating to transfer anterior outward pressure applied to the anterior mechanical actuator through the mechanical linkage to the posterior mechanical compensator to cause the posterior mechanical compensator to move inwardly relative to the receptacle;
wherein the mechanical linkage includes a gearing mechanism whereby an anterior component of inward movement of the posterior mechanical compensator exceeds an anterior component of outward movement of the anterior mechanical actuator.
